(19) United States
(12) Patent Application Publication
(10) Pub. No.: US 2009/0246770 A1
(43) Pub. Date: Oct. 1, 2009

(54) GLYCEMIC CONTROL FOR REDUCTION OF CARDIOVASCULAR DISEASE RISK IN DIABETIC PATIENTS EXPRESSING HAPTOGLOBIN 2-2

(76) Inventor: Andrew Levy, Haifa (IL)

Correspondence Address: Pearl Cohen Zedek Latzer, LLP, 1500 Broadway, 12th Floor, New York, NY, 10036, US

(21) Appl. No.: 12/262,384

(22) Filed: Oct. 31, 2008

Related U.S. Application Data
(60) Provisional application No. 60/996,105, filed on Nov. 1, 2007.

(51) Int. Cl. C12Q 1/68

(52) U.S. Cl. 435/6

(57) ABSTRACT

This invention relates to methods of reducing risk of developing cardiovascular complications in diabetic patients. Specifically, the invention relates to the use of haptoglobin genotyping for determining the importance of maintaining tight glycemic control in diabetic subjects expressing Hp 2-2 allele.

### CROSS-REFERENCE TO RELATED APPLICATION

This application claims priority to U.S. provisional application Ser. No. 60/996,105, filed Nov. 1, 2007, the contents of which is incorporated herein by reference in its entirety.

### FIELD OF INVENTION

This invention is directed to methods of reducing risk of developing cardiovascular complications in diabetic patients. Specifically, the invention is directed to the use of haptoglobin (Hp) genotyping for determining the importance of maintaining tight glycemic control in diabetic subjects expressing Hp2-2 allele.

### BACKGROUND OF THE INVENTION

The haptoglobin (Hp) gene is polymorphic in man with two classes of alleles denoted 1 and 2. Several cross sectional and retrospective analysis have suggested that the Hp genotype may be a major determinant of susceptibility to diabetic cardiovascular disease (CVD).

Likewise, extensive studies, including the Diabetes Control and Complications Trial (DCCT) (See DCCT Research Group: The Effect Of Intensive Treatment Of Diabetes On The Development And Progression Of Long-Term Complications Of Insulin-Dependent Diabetes Mellitus. New England Journal of Medicine, 329: 978-986, 1993), the Stockholm Diabetes Intervention. Study (See Reichard P, Phil M: Mortality and Treatment Side Effects During Long-term Intensified Conventional Insulin Treatment in the Stockholm Diabetes Intervention Study. Diabetes, 43: 313-317, 1994), and the United Kingdom Prospective Diabetes Study (See UK Prospective Diabetes Study Group: Effect of Intensive Blood Glucose Control With Metformin On Complications In Patients With Type 2 Diabetes (UKPDS 34). Lancet, 352: 837-853, 1998), have repeatedly demonstrated that the most effective way to prevent the long term complications of diabetes is by strictly maintaining blood glucose (BG) levels within a normal range using intensive insulin therapy.

It has been well known for more than twenty years that glycosylated hemoglobin (HbA_{1c}) is a marker for the glycemic control of individuals with diabetes mellitus (DM, type I or type II). Numerous researchers have investigated this relationship and have found that glycosylated hemoglobin generally reflects the average BG levels of a patient over the previous two months. Since in the majority of patients with diabetes the BG levels fluctuate considerably over time, it was suggested that the real connection between integrated glucose control and HbA_{1c} would be observed only in patients known to be in stable glucose control over a long period of time.

Guidelines were developed indicating that an HbA_{1c} of 7% corresponds to a mean BG of 8.3 mM (150 mg/dl), an HbA_{1c} of 9% corresponds to a mean BG of 11.7 mM (210 mg/dl), and a 1% increase in HbA_{1c} corresponds to an increase in mean BG of 1.7 mM (30 mg/dl). The DCCT also suggested that because measuring the mean BG directly is not practical, one could assess a patient's glycemic control with a single, simple test, namely HbA_{1c}. However, studies clearly demonstrate that HbA_{1c} is not sensitive to hypoglycemia.

The unmet challenge of achieving tight glycemic control is due, in part, to the shortcomings of frequent BG monitoring, as well as the lack of predictability regarding hyperglycemia. Accordingly, there is a need for better prediction the importance of maintaining tight glycemic control in diabetic subjects.

### SUMMARY OF THE INVENTION

In one embodiment, the invention provides a method of reducing the risk of a diabetic subject developing a cardiovascular disease (CVD), comprising the steps of obtaining a biological sample from the subject; determining the subject's haptoglobin allelic genotype; and controlling the level of HbA_{1c} below a predetermined threshold in a subject expressing the Hp-2-2 genotype.

In another embodiment, provided herein is a method of determining prognosis for a diabetic subject, to benefit from a glycemic control comprising the step of obtaining a biological sample from the subject; and determining the subject's haptoglobin allelic genotype, whereby a subject expressing the Hp-2-2 genotype will benefit from a glycemic control.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows the cardiovascular event rate when HbA_{1c} is maintained above or below the 7.0% threshold in diabetic patients according to their haptoglobin genotype.

### DETAILED DESCRIPTION OF THE INVENTION

This invention relates in one embodiment to methods of reducing risk of developing cardiovascular complications in patients with diabetes mellitus (DM). In another embodiment, provided herein are methods for the use of haptoglobin genotyping for determining the importance of maintaining tight glycemic control in diabetic subjects expressing the Hp 2 allele.

In one embodiment, the optimal utilization of health care resources for cardiovascular risk factor modification should be focused on DM individuals with the Hp 2-2 genotype. Benefit from tight glycemic control only in a subset of the DM cohort defined by the Hp 2-2 genotype explains in another embodiment, the inability to show a benefit from tight glycemic control on reducing cardiovascular events in the entire DM cohort in multiple prior clinical studies.

In another embodiment, reducing the risk of developing cardiovascular disease (CVD) as described herein, refers to a first cardiovascular (CV) event with clinical findings of nonfatal myocardial infarction or stroke; or death due to CV disease; subclinical myocardial infarction identified on an annual electrocardiogram; angina confirmed by stress test or angiography; or the need for revascularization with angioplasty or coronary artery bypass in other discrete embodiments.

Accordingly and in one embodiment, provided herein is a method of reducing the risk of a diabetic subject developing a cardiovascular disease, comprising the steps of obtaining a biological sample from the subject; determining the subject's haptoglobin allelic genotype; and controlling the level of HbA_{1c} below a predetermined threshold in a subject expressing the Hp 2-2 genotype.

In another embodiment, the term “glycemic control” refers to guidelines that were developed indicating that an HbA_{1c} of 7% corresponds to a mean BG of 8.3 mM (150 mg/dl), an HbA_{1c} of 9% corresponds to a mean BG of 11.7 mM (210 mg/dl), and a 1% increase in HbA_{1c} corresponds to an increase in mean BG of 1.7 mM (30 mg/dl). The DCCT also suggested that because measuring the mean BG directly is not practical, one could assess a patient's glycemic control with a single, simple test, namely HbA_{1c}.

Most type 1 diabetes patients are managed according to standard American Diabetes Association (ADA) goals, which currently recommend conventional therapy to achieve HbA1c levels of <7%. Other groups such as the American College of Endocrinology recommend tighter HbA1c values of ≦6.5% to minimize risk of microvascular and macrovascular complications. Accordingly, in another embodiment, the threshold below which the HbA_{1c} is maintained in subjects exhibiting the Hp 2-2 allele, to reduce the risk of CVD as described herein, is 6.5%.

Glycated hemoglobin (HbA_{1c}) is a biomarker used to measure blood glucose control. Glucose is carried in the blood stream and becomes attached to the hemoglobin molecule. As a result of this attachment, changes occur which can be measured to estimate the average glucose level for the life of the hemoglobin molecule. HbA_{1c} measurement is the primary measure of glucose control used by the FDA to determine the efficacy of drug candidates in diabetics.

In one embodiment, maintaining glycemic control refers to HbA_{1c} testing twice a year in subjects meeting glycemic goals, and quarterly for subjects who do not. In another embodiment, maintaining tight glycemic control is done using the methods described herein as well as taking a thorough medical history, physical exam and a family history of diabetes or other endocrine disorders; or performing a cardiac evaluation; assessing diet and medications that may affect glucose levels; measuring HbA_{1c} levels, fasting lipid profile, albumin levels in the urine, and serum creatinine in children in the presence of proteinuria; and performing urinalysis for ketones, protein, and sediment in other discrete embodiments. In one embodiment Haptoglobin genotyping as described in the methods provided herein, is done before, during or following diagnosis of diabetes. In another embodiment, once a subject is classified as a Hp-2-2 carrier, tight glycemic control is done using the criteria described hereinabove at a higher frequency for both subjects meeting glycemic goals, and for subjects who do not, while in one embodiment the goal is 7.0% or 6.5% in another embodiment.

In one embodiment, lowering the HbA_{1c}, to maintain glycemic control in accordance with the methods described herein, comprises contacting the subject with a composition comprising biguanides, referring to agents capable of shutting off the liver's excess glucose production, such as metformin in one embodiment, or sulphonylureas, referring to second- and third generation agents capable of stimulating the pancreas to make more insulin, or meglitinides, referring to agents capable of stimulating the pancreas to make more insulin, or α-glucosidase inhibitors, referring to agents capable of slowing absorption of carbohydrates in the intestine, PPARγ-agonists, referring to agents capable of enhancing glucose disposal in a variety of insulin-resistant states in humans and animals, GLP-1 agonists, referring to agents capable of increasing cellular release of glucagon-like peptide (GLP-1), thereby inducing glucose-dependent insulin secretion in the pancreas; DPP-IV inhibitors, referring to agents capable of inhibiting the enzyme responsible for GLP-1 breakdown; thiazolidinediones, referring to agents capable of increasing the body's sensitivity to insulin, insulin or their combination in other discrete embodiments.

In a further embodiment, the vascular complication is a macrovascular complication. In another embodiment, the vascular disease is a chronic heart failure. In another embodiment, the vascular disease is cardiovascular death. In another embodiment, the vascular disease is stroke. In another embodiment, the vascular disease is myocardial infarction. In another embodiment, the vascular disease is coronary angioplasty associated restenosis. In another embodiment, the vascular disease is fewer coronary artery collateral blood vessels and myocardial ischemia in other embodiments. In another embodiment, the vascular complication is a microvascular complication, such as diabetic neuropathy in one embodiment. In another embodiment, the microvascular disease is diabetic nephropathy or diabetic retinopathy in yet another embodiment.

Microvascular disease may be characterized in one embodiment, by an unevenly distributed thickening (or hyalinization) of the intima of small arterioles, due in another embodiment, to the accumulation of type IV collagen in the basement membrane, or microaneurisyms of the arterioles, which compromises the extent of the maximal arteriolar dilation that can be achieved and impairs the delivery of nutrients and hormones to the tissues, or to remove waste in another embodiment. The vasculature distal to the arterioles may also be affected in one embodiment, such as by increased capillary basement membrane thickening, abnormalities in endothelial metabolism, or via impaired fibrinolysis, also resulting in reduced delivery of nutrients and hormones to the tissues, or waste removal in another embodiment.

According to various typical embodiments of the method of the present invention, determining the haptoglobin phenotype of a subject is effected by any one of a variety of methods including, but not limited to, a signal amplification method, a direct detection method and detection of at least one sequence change. These methods determine a phenotype indirectly, by determining a genotype. As will be explained hereinbelow, determination of a haptoglobin phenotype may also be accomplished directly by analysis of haptoglobin gene products.

Haptoglobin is inherited by two co-dominant autosomal alleles situated on chromosome 16 in humans, these are Hp1 and Hp2. There are three phenotypes Hp1-1, Hp2-1 and Hp2-2. Haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles. The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined in one embodiment, from 10 μl of plasma by gel electrophoresis.

In certain embodiments, the method for determining haptoglobin genotype can be a signal amplification method, a direct detection method, detection of at least one sequence change, an immunological method or a combination thereof.

A signal amplification method according to various preferred embodiments of the present invention may amplify, for example, a DNA molecule or an RNA molecule. Signal amplification methods which might be used as part of the present invention include, but are not limited to PCR, LCR (LAR), Self-Sustained Synthetic Reaction (3SR/NASBA) or a Q-Beta (Qβ) Replicase reaction.

Polymerase Chain Reaction (PCR): The polymerase chain reaction (PCR), refers in one embodiment to a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, in one embodiment, they are said to be “PCR-amplified.”

Ligase Chain Reaction (LCR or LAR): The ligase chain reaction [LCR; referred to, in another embodiment as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed in one embodiment and DNA ligase is added to the mixture. Provided that there is complete complementarity at the junction, ligase will covalently link each set of hybridized molecules. In another embodiment of LCR, two probes are ligated together only when they base-pair with sequences in the target sample, without gaps or mismatches. Repeated cycles of denaturation, and ligation amplify a short segment of DNA. LCR has is used in combination with PCR in one embodiment, to achieve enhanced detection of single-base changes. In another embodiment, because the four oligonucleotides used in this assay can pair to form two short ligatable fragments, there is the potential for the generation of target-independent background signal. The use of LCR for mutant screening is limited in another embodiment, to the examination of specific nucleic acid positions.

Self-Sustained Synthetic Reaction (3SR1NASBA): The self-sustained sequence replication reaction (3SR) refers in one embodiment, to a transcription-based in vitro amplification system that can exponentially amplify RNA sequences at a uniform temperature. The amplified RNA is utilized in certain embodiments, for mutation detection. In an embodiment of this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ.) Replicase: In one embodiment of the method, a probe which recognizes the sequence of interest is attached to the replicatable RNA template for Qβ. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

In another embodiment, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

In another embodiment, the methods provided herein for reducing the risk of a diabetic subject developing a cardiovascular disease (CVD), comprising the steps of obtaining a biological sample from the subject; determining the subject's haptoglobin allelic genotype; and controlling the level of HbA1c below a predetermined threshold in a subject expressing the Hp-2-2 genotype, or in another embodiment, for determining prognosis for a diabetic subject, to benefit from a glycemic control is effected by a direct detection method such as a cycling probe reaction (CPR), or a branched DNA analysis, or a combination thereof in other embodiments.

The direct detection method according to one embodiment is a cycling probe reaction (CPR) or a branched DNA analysis. When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

In another embodiment, the methods provided herein for reducing the risk of a diabetic subject developing a cardiovascular disease, comprising the steps of obtaining a biological sample from the subject; determining the subject's haptoglobin allelic genotype; and controlling the level of HbA1c below a predetermined threshold in a subject expressing the Hp-2-2 genotype, or in another embodiment, for determining prognosis for a diabetic subject, to benefit from a glycemic control, is effected by at least one sequence change, which employs in one embodiment a restriction fragment length polymorphism (RFLP analysis), or an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF) or their combination in other embodiments.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Determination of a haptoglobin phenotype may, as is further exemplified in the Examples section that hereinbelow, may be accomplished directly in one embodiment, by analyzing the protein gene products of the haptoglobin gene, or portions thereof. Such a direct analysis is often accomplished using an immunological detection method. In one embodiment, the methods and systems provided herein for providing a method for reducing cardiovascular disease risk in a diabetic subject to benefit from improving glycemic control, comprising the steps of: obtaining a biological sample from a subject; determining the haptoglobin (Hp) genotype in the biological sample by an immunological detection method, such as is a radio-immunoassay (RIA) in one embodiment, or an enzyme linked immunosorbent assay (ELISA), a sandwich ELISA, a western blot, an immunohistochemical analysis, or fluorescence activated cell sorting (FACS), or a combination thereof in other embodiments.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with ¹²⁵I) immobilized on a precipitable carrier such as agarose beads. The number of counts in the precipitated pellet is proportional to the amount of substrate. In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Sandwich ELISA measures the amount of antigen between two layers of antibodies (i.e. capture and detection antibody). The antigen to be measured must contain at least two antigenic sites capable of binding to antibody, since at least two antibodies act in the sandwich. Either monoclonal or polyclonal antibodies can be used as the capture and detection antibodies in Sandwich ELISA systems. Monoclonal antibodies recognise a single epitope that allows fine detection and quantification of small differences in antigen. A polyclonal is often used as the capture antibody to pull down as much of the antigen as possible. The advantage of Sandwich ELISA is that the sample does not have to be purified before analysis, and the assay can be very sensitive (up to 2 to 5 times more sensitive than direct or indirect).

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

Cardiovascular disease (CVD) is the most frequent, severe and costly complication of type 2 diabetes. It is the leading cause of death among patients with type 2 diabetes regardless of diabetes duration. In one embodiment, allelic polymorphism contributes to the phenotypic expression of CVD in diabetic subjects. In another embodiment, the methods of the invention are used in the treatment of CVD in diabetic subjects, or in another embodiment, in the determination of preferred course of treatment.

The term “myocardial infarct” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infarct” refers to the death of a certain segment of the heart muscle (myocardium), which in one embodiment, is the result of a focal complete blockage in one of the main coronary arteries or a branch thereof. In one embodiment, subjects which were formerly diagnosed as having severe, stable or unstable angina pectoris, are treated according to the of the invention, upon determining these subjects carry the Hp-2 allele and are diabetic.

The term “ischemia-reperfusion injury” refers in one embodiment to a list of events including: reperfusion arrhythmias, microvascular damage, reversible myocardial mechanical dysfunction, and cell death (due to apoptosis or necrosis). These events may occur in another embodiment, together or separately. Oxidative stress, intracellular calcium overload, neutrophil activation, and excessive intracellular osmotic load explain in one embodiment, the pathogenesis and the functional consequences of the inflammatory injury in the ischemic-reperfused myocardium. In another embodiment, a close relationship exists between reactive oxygen species and the mucosal inflammatory process.

In one embodiment, the methods described herein are followed by the administration with other therapeutical agents. Representative agents that can be used in combination with the methods of the invention are agents used to treat diabetes such as insulin and insulin analogs (e.g. LysPro insulin); GLP-1 (7-37) (insulinotropin) and GLP-1 (7-36)-NH₂; biguanides: metformin, phenformin, buformin; alpha2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPARγ agonists; fatty acid oxidation inhibitors: clomoxir, etomoxir; alpha.-glucosidase inhibitors: acarbose, miglitol, emiglitate, voglibose, MDL-25,637, camiglibose, MDL-73,945; beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. NAGLIVAN) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (SYMLIN), AC2993, glycogen phosphorylase inhibitor and nateglinide. Any combination of agents can be administered as described hereinabove.

In another embodiment, lowering the level of HbA_{1c} following the Hp genotyping according to the methods described herein, is done by contacting the subject with a composition comprising metformin, a sulphonylurea, Repaglinide, an α-glucosidase inhibitor, a PPARγ-agonist, insulin or their combination, each a discrete embodiment of the methods described herein. In another embodiment, diabetic patients treated with pyridoxal-5′-phosphate (P5P), alone or in combination with an ACE inhibitor have improved metabolic function.

The renin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme renin from the juxtaglomerular cells in the kidney activates in another embodiment, the renin-angiotensin-aldosterone system (RAAS), acting on a naturally-occurring substrate, angiotensinogen, to release in another embodiment, a decapeptide, angiotensin I. Angiotensin converting enzyme (“ACE”) cleaves in one embodiment, the secreated decapeptide, producing an octapeptide, angiotensin II, which is in another embodiment, the primary active species of the RAAS system. Angiotensin II stimulates in one embodiment, aldosterone secretion, promoting sodium and fluid retention, inhibiting renin secretion, increasing sympathetic nervous system activity, stimulating vasopressin secretion, causing a positive cardiac inotropic effect or modulating other hormonal systems in other embodiments.

A representative group of ACE inhibitors used in one embodiment in the compositions used for lowering HbA_{1c} following the determination that a diabetic subject expresses the Hp2-2 allele, consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, I5B2, indolapril, ketomethylureas, KRI-1177, KRI-1230, L-681176, libenzapril, MCD, MDL-27088, MDL-27467A, moveltipril, MS-41, nicotianamine, pentopril, phenacein, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1-(−(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the compositions provided herein are used for the lowering of HbA_{1c} in a diabetic subject, may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In some embodiments, the compositions and methods provided herein permit direct application to the site where it is needed. In the practice of the methods provided herein, it is contemplated that virtually any of the compositions provided herein can be employed.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein are suitable for oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical, or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with one or more therapeutic agents. These agents are in other embodiments, age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

In one embodiment, the compositions described herein, are used in the methods described herein. Accordingly and in another embodiment, provided herein are compositions comprising a HbA_{1c} lowering agent, and an effective amount of a composition comprising glutathione peroxidase or its isomer, metabolite, and/or salt therefore. In one embodiment, the glutathione peroxidase or its mimetic, isomer, metabolite, and/or salt therefore is 3,3-dimethyl-benzisoselenazoline.

In one embodiment, the methods provided herein, using the compositions provided herein, further comprise contacting the subject with one or more additional agent, which is not a HbA1c-lowering agent. In another embodiment, the one or more additional agent, which is a HbA1c-lowering agent, is an aldosterone inhibitor. In another embodiment, the additional agent is an angiotensin-converting anzyme. In another embodiment, the additional agent is an antioxidant. In another embodiment, the additional agent is an angiotensin receptor AT₁ blocker (ARB). In another embodiment, the additional agent is an angiotensin II receptor antagonist. In another embodiment, the additional agent is a calcium channel blocker. In another embodiment, the additional agent is a diuretic. In another embodiment, the additional agent is digitalis. In another embodiment, the additional agent is a beta blocker. In another embodiment, the additional agent is a statin. In another embodiment, the additional agent is a cholestyramine or in another embodiment, the additional agent is a combination thereof.

In one embodiment, the additional therapeutic agent used in the methods and compositions described herein is a statin. In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

Statins inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease.

Statins used in the compositions and methods of the invention are lovastatin (referred to as mevinolin in one embodiment, or monacolin-K in another embodiment), compactin (referred to as mevastatin in one embodiment, or ML-236B in another embodiment), pravastatin, atorvastatin (Lipitor) rosuvastatin (Crestor) fluvastatin (Leschol), simvastatin (Zocor), cerivastatin. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

In one embodiment, the additional agent may be an anti-dyslipidemic agent such as (i) bile acid sequestrants such as, cholestyramine, colesevelem, colestipol, dialkylaminoalkyl derivatives. of a cross-linked dextran; Colestid™; LoCholest™; and Questran™, and the like; (ii) HMG-CoA reductase inhibitors such as atorvastatin, itavastatin, fluvastatin, lovastatin, pravastatin, rivastatin, rosuvastatin, simvastatin, and ZD-4522, and the like; (iii) HMG-CoA synthase inhibitors; (iv) cholesterol absorption inhibitors such as stanol esters, beta-sitosterol, sterol glycosides such as tiqueside; and azetidinones such as ezetimibe, vytorin, and the like; (v) acyl coenzyme A-cholesterol acyl transferase (ACAT) inhibitors such as avasimibe, eflucimibe, KY505, SMP 797, and the like; (vi) CETP inhibitors such as JTT 705, torcetrapib, CP 532,632, BAY63-2149, SC 591, SC 795, and the like; (vii) squalene synthetase inhibitors; (viii) anti-oxidants such as probucol, and the like; (ix) PPAR.alpha. agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid™, Lopid™ and Tricor™, and the like; (x) FXR receptor modulators such as GW 4064, SR 103912, and the like; (xi) LXR receptor such as GW 3965, T9013137, and XTC0179628, and the like; (xii) lipoprotein synthesis inhibitors such as niacin; (xiii) renin angiotensin system inhibitors; (xiv) PPAR o partial agonists; (xv) bile acid reabsorption inhibitors, such as BARI 1453, SC435, PHA384640, S892.1, AZD7706, and the like; (xvi) PPAR.delta. agonists such as GW 501516, and GW 590735, and the like; (xvii) triglyceride synthesis inhibitors; (xviii) microsomal triglyceride transport (MTTP) inhibitors, such as inplitapide, LAB687, and CP346086, and the like; (xix) transcription modulators; (xx) squalene epoxidase inhibitors; (xxi) low density lipoprotein (LDL) receptor inducers; (xxii) platelet aggregation inhibitors; (xxiii) 5-LO or FLAP inhibitors; and (xiv) niacin receptor agonists.

In one embodiment, the additional agent administered as part of the compositions, used in the methods provided herein, is an anti-platelet agents (or platelet inhibitory agents). The term anti-platelet agents (or platelet inhibitory agents), refers in one embodiment to agents that inhibit platelet function by inhibiting the aggregation, or by adhesion or granular secretion of platelets in other embodiments. In another embodiment, the anti-platelet agents used in the compositions described herein include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. In another embodiment, the anti-platelet agent is IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof. In another embodiment, the term anti-platelet agents (or platelet inhibitory agents), refers to ADP (adenosine diphosphate) receptor antagonists, which is in one embodiment, an antagonists of the purinergic receptors P₂Y₁ and P₂Y₁₂. In one embodiment, P₂Y₁₂ receptor antagonists is ticlopidine, clopidogrel, or their combination and pharmaceutically acceptable salts or prodrugs thereof.

In another embodiment, the additional agent administered as part of the compositions, used in the methods provided herein, is an anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (ix) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XEN010, and the like; and (xi)-alpha 2 agonists such as lofexidine, tiamenidine, moxonidine, rilmenidine and guanobenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses Aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the Aldosterone, even when there are spare (unbound) Aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplerenone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a HbA_{1c} lowering agent, wherein the aldosterone antagonist is epoxymexrenone, or eplerenone, dihydrospirorenone, 2,2; 6,6-diethlylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In one embodiment, the antioxidants include small-molecule antioxidants and antioxidant enzymes. Suitable small-molecule antioxidants include, in another embodiment, hydralazine compounds, glutathione, vitamin C, vitamin E, cysteine, N-acetyl-cysteine, .beta.-carotene, ubiquinone, ubiquinol-10, tocopherols, coenzyme Q, and the like. Suitable antioxidant enzymes include in one embodiment superoxide dismutase, catalase, glutathione peroxidase, or a combination thereof. Suitable antioxidants are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996.

In one embodiment, the therapeutic value of the primary agents described above in the compositions provided herein, can be further augmented by administration in conjunction with recognized antioxidant free radical trapping compounds such as α-tocopherol, edaravone or other co-agents previously recognized as adjunts which facilitate in vivo capability to inhibit lipid peroxidation.

In one embodiment agents which function to supplement the chain-breaking antioxidant property of vitamin E are ubiquinol, or seleno-amino acids and sulfhydryl compounds (e.g., glutathione, sulfhydryl proteins, cysteine and methionine) in other embodiments. Other substances in this general group include in other embodiments: butylated hydroxytoluene (BHT), butylated hydroxyanisole (BHA), propyl gallate (PG), dodecylgallate, tert-butylhydroquinone (TBHQ), dihydrolipoic acid, prostaglandin B₁ oligomers (also known as polymeric 15-keto prostaglandin B or PGBX), 2-aminomethyl-4-tert-butyl-6-iodophenol, 2-aminomethyl-4-tert-butyl-6-propionylphenol, 2,6-di-tert-butyl-4-[2′-thenoyl]phenol, N,N′-diphenyl-p-phenylenediamine, ethoxyquin, probucol and its derivative such as AGI-1067, 5-[[3,5-bis(1,1-dimethylethyl)-4-hydroxyphen-yl]methylene]-3-(dimethylamino)-4-thiazolidinone (LY221068), 5-[[3,5-bis(1,1-dimethylethyl)-4-hydroxyphenyl]meth-ylene]-3-(methylamino)-4-thiazolidinone (LY269415), D-myoinositol-1.2.6-trisphosphate, nordihydroguaiaretic acid, deferoxamine mesylate, tirilazad mesylate (U-74006F), derivative of tirilazad in which the steroid portion of the chemical structure has been replaced with the tetramethyl chroman portion of d-α-tocopherol (U78517F), trimetazidine, N,N′-dimethylthiourea, 2-(2-hydroxy-4-methylphenyl) aminothiazole-hydrochloride, or 2-L-oxothiazolidine.

In another embodiment, Thioctic acid, also known as α-lipoic acid, is used as an antioxidant in the compositions and methods provided herein, including its sodium salt and ethylenediamine derivatives. In one embodiment, antioxidants and free radical trapping substances used in the compositions and methods provided herein, are plant (e.g., vegetable) active ingredients. This category, includes in one embodiment parthenolide, or lycopene, genistein, quercetin, morin, curcumin, apigenin, sesamol, chlorogenic acid, fisetin, ellagic acid, quillaia saponin, capsaicin, ginsenoside, silymarin, kaempferol, ginkgetin, bilobetin, isoginkgetin, isorhamnetin, herbimycin, rutin, bromelain, levendustin A, orerbstatin in other embodiments

In addition to a direct action on arteries and arterioles, angiotensin II (AII), is one of the most potent endogenous vasoconstrictors known, exerts in one embodiment, stimulation on the release of aldosterone from the adrenal cortex. Therefore, the renin-angiotensin system, (RAAS) by virtue of its participation in the control of renal sodium handling, plays an important role in cardiovascular hemeostasis.

In another embodiment, the angiotensin II receptor antagonist used in the compositions and methods of the invention is losartan, irbesartan, eprosartan, candesartan, valsartan, telmisartan, zolasartin, tasosartan or a combination thereof. Examples of angiotensin II receptor antagonists used in the compositions and methods of the invention are in one embodiment biphenyltetrazole compounds or biphenylcarboxylic acid compounds or CS-866, losartan, candesartan, valsartan or irbesartan in other embodiments. In one embodiment, where the above-mentioned compounds have asymmetric carbons, the angiotensin II receptor antagonists of the compositions and methods used in the present invention are optical isomers and mixtures of said isomers. In one embodiment, hydrates of the above-mentioned compounds are also included.

In one embodiment, cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca²⁺-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in cystolic pressure.

In one embodiment, calcium channel blockers, are amlodipine, aranidipine, barnidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, veraparmil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, angiotensin II receptor blocker (ARB) are used in the compositions and methods of the invention. Angiotensin II receptor blocker (ARB) refers in one embodiment to a pharmaceutical agent that selectively blocks the binding of AII to the AT₁ receptor. ARBs provide in another embodiment, a more complete blockade of the RAAS by preventing the binding of AII to its primary biological receptor (AII type 1 receptor [AT₁]).

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglycate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In another embodiment, the additional agent administered as part of the compositions, used in the methods provided herein, is a glycation inhibitor, such as pimagedine hydrochloride in one embodiment, or ALT-711, EXO-226, KGR-1380, aminoguanidine, ALT946, pyratoxanthine, N-phenacylthiazolium bromide (ALT766), pyrrolidinedithiocarbamate or their combination in yet another embodiment.

In one embodiment, the methods and compositions described hereinabove are used in a method of determining prognosis for a diabetic subject, to benefit from a glycemic control comprising the step of obtaining a biological sample from the subject; and determining the subject's haptoglobin allelic genotype, whereby a subject expressing the Hp-2-2 genotype will benefit from a glycemic control.

In another embodiment, the glycemic control, comprises lowering the level of HbA1c to below the predetermined threshold. In certain embodiments, the improved glycemic control is demonstrated by improved HbA_{1c} levels after treatment compared with baseline levels prior to treatment. In one embodiment, the improved HbA_{1c} levels are measured by a statistically significant decline in HbA_{1c} levels. In another embodiment, administration of the compositions described herein are made to a mammal with impaired glucose tolerance or with early or late stage diabetes having an HbA_{1c} level ranging from normal to elevated prior to treatment. In one embodiment, the mammal may have an HbA_{1c} level preferably of above than about 8.0 prior to treatment.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

Over 3000 individuals 55 years of age or older with DM were recruited from 47 primary health care clinics of the Clalit Health Plan in Northern Israel and a Hp genotype was obtained on all of these individuals. The prevalence of CVD at baseline was 25%. Patients were followed for two years, for the primary composite outcome of the study which was incident non-fatal myocardial infarction, stroke and CV death.

### Example 1

### Hp2-2 Genotype is Associated with a Highly Significant Increase in CVD

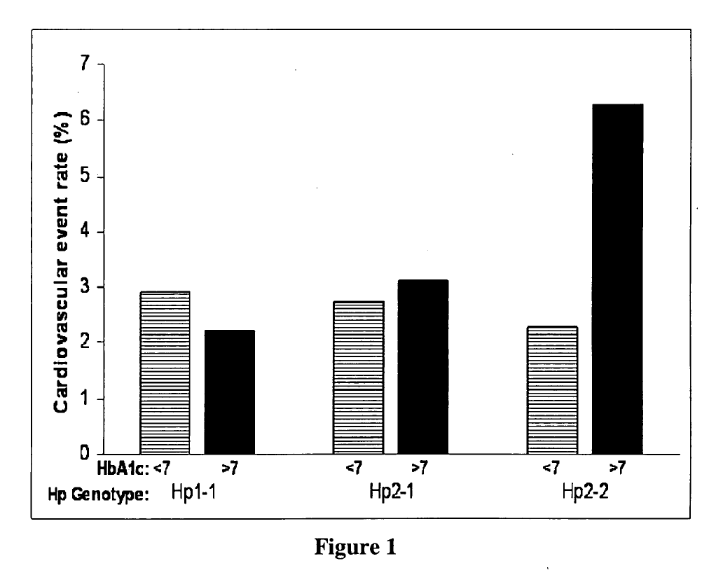

The Hp 2-2 genotype was found to be associated with a highly significant increase in the incidence of myocardial infarction, stroke and CV death (See e.g. FIG. 1). Moreover, after stratification of patients by baseline HbA_{1c} to those above and below 7.0, representing inadequate or adequate glycemic control as currently recommended by the AHA and ADA, only in Hp 2-2 individuals was poor glycemic control found to be associated with an increased risk of major cardiovascular events.

The following Table provides the number of cardiovascular events and patients stratified by Hp type and whether they were above or below a certain HbA_{1c} cutoff. Note the only significant benefit to HbA_{1c} lowering was found in Hp 2-2 with lowering below 7.0 as compared to above 7.0. No other group was significant (i.e., p<0.05).

| HbA1c cut | Hp 1-1 | | Hp 2-1 | | Hp 2-2 | |
|---|---|---|---|---|---|---|
| off | < | > | < | > | < | > |
| 6.5 | 2/91 | 3/180 | 7/350 | 13/802 | 11/403 | 36/937 |
| | (2.2%) | (1.7%) | (2%) | (1.6%) | (2.7%) | (3.8%) |
| 7 | 3/142 | 2/129 | 8/536 | 12/616 | 16/651 | 31/689 |
| | (2.1%) | (1.5%) | (1.5%) | (1.9%) | (2.5%) | (4.5%) |
| 7.5 | 4/183 | 1/88 | 9/734 | 11/418 | 25/868 | 22/472 |
| | (2.2%) | (1.1%) | (1.2%) | (2.6%) | (2.9%) | (4.7%) |
| 8 | 4/210 | 1/61 | 13/866 | 7/286 | 33/1029 | 14/311 |
| | (1.9%) | (1.6%) | (1.5%) | (2.4%) | (3.2%) | (4.5%) |

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of reducing the risk of a diabetic subject developing a cardiovascular disease, comprising the steps of obtaining a biological sample from the subject; determining the subject's haptoglobin allelic genotype; and controlling the level of HbA1c below a predetermined threshold in a subject expressing the Hp-2-2 genotype.

2. The method of claim 1, wherein said step of determining said haptoglobin genotype is effected by a method selected from a signal amplification method, a direct detection method, detection of at least one sequence change, immunological method or a combination thereof.

3. The method of claim 3, wherein said signal amplification method amplifies a molecule selected from the group consisting of a DNA molecule and an RNA molecule.

4. The method of claim 3, wherein said signal amplification method is selected from the group consisting of PCR, LCR (LAR), Self-Sustained Synthetic Reaction (3SR/NASBA) and Q-Beta (Qβ) Replicase reaction.

5. The method of claim 3, wherein said direct detection method is selected from the group consisting of a cycling probe reaction (CPR) and a branched DNA analysis.

6. The method of claim 3, wherein said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

7. The method of claim 3, wherein step of determining said haptoglobin genotype is effected by an immunological detection method.

8. The method of claim 7, wherein said immunological detection method is a radio-immunoassay (RIA), an enzyme linked immunosorbent assay (ELISA), a sandwhich immunoassay, a western blot, an immunohistochemical analysis, or fluorescence activated cell sorting (FACS).

9. The method of claim 1, whereby the diabetic subject exhibits type II diabetes.

10. The method of claim 1, whereby the reducing the risk comprises determining the importance of reducing oxidative stress.

11. The method of claim 1, wherein the HbA_{1c} level is below about 7.0%.

12. The method of claim 1, wherein the HbA_{1c} level is below about 6.5%.

13. The method of claim 1, whereby the cardiovascular disease is angina, myocardial infarct, peripheral vascular disease, cerebrovascular disease or a combination thereof.

14. A method of determining prognosis for a diabetic subject, to benefit from a glycemic control comprising the step of obtaining a biological sample from the subject; and determining the subject's haptoglobin allelic genotype, whereby a subject expressing the Hp-2-2 genotype will benefit from a glycemic control.

15. The method of claim 14, wherein the glycemic control comprises lowering the level of HbA1c to below the predetermined threshold

16. The method of claim 14, wherein lowering the level of HbA1c is done by contacting the subject with a composition comprising: a biguanide, a sulphonylureas, a meglitinides, an α-glucosidase inhibitor, a PPARγ-agonists, a GLP-1 agonist, a DPP-IV inhibitor, an insulin or their combination.

17. The method in claim 15 wherein the lowering of the level of Hb1Ac is done by exercise, diet or their combination.

18. The method of claim 15, wherein the HbA_{1c} level is below about 7.0%.

19. The method of claim 15, wherein the HbA_{1c} level is below about 6.5%.

20. The method of claim 14, wherein said step of determining said haptoglobin genotype is effected by a method selected from a signal amplification method, a direct detection method, detection of at least one sequence change, immunological method or a combination thereof.

21. The method of claim 20, wherein said signal amplification method amplifies a molecule selected from the group consisting of a DNA molecule and an RNA molecule.

22. The method of claim 20, wherein said signal amplification method is selected from the group consisting of PCR, LCR (LAR), Self-Sustained Synthetic Reaction (3SR/NASBA) and Q-Beta (Qβ) Replicase reaction.

23. The method of claim 20, wherein said direct detection method is selected from the group consisting of a cycling probe reaction (CPR) and a branched DNA analysis.

24. The method of claim 20, wherein said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

25. The method of claim 14, wherein step of determining said haptoglobin genotype is effected by an immunological detection method.

26. The method of claim 25, wherein said immunological detection method is a radio-immunoassay (RIA), an enzyme linked immunosorbent assay (ELISA), a sandwhich immunoassay, a western blot, an immunohistochemical analysis, or fluorescence activated cell sorting (FACS).

27. The method of claim 14, whereby the diabetic subject exhibits type II diabetes.
